(19) United States
(12) Patent Application Publication
(10) Pub. No.: US 2024/0325362 A1
(43) Pub. Date: Oct. 3, 2024

(54) NOVEL THERAPEUTIC METHOD FOR ENHANCING MITOCHONDRIAL FUNCTION, TREATING MITOCHONDRIAL DISEASES THEREBY, AND COMPOUNDS USED THEREIN

(71) Applicant: STANDIGM INC., Seoul (KR)

(72) Inventors: Minyoung SO, Ann Arbor, MI (US); Jinhan KIM, Seoul (KR); Sangok SONG, Seongnam-si, Gyeonggi-do (KR); Sanghyung JIN, Seoul (KR)

(21) Appl. No.: 18/262,721

(22) PCT Filed: Jul. 16, 2021
(86) PCT No.: PCT/KR2021/009180

§ 371 Date: Jul. 24, 2023

Related U.S. Application Data
(60) Provisional application No. 63/057,992, filed on Jul. 29, 2020.

(51) Int. Cl. A61K 31/4422; A61K 31/403; A61K 31/455; A61K 31/495; A61K 31/496; A61K 31/519; A61K 31/5377; A61K 31/565; A61K 31/573; A61K 31/675; A61P 3/00

(52) U.S. Cl. CPC A61K 31/4422; A61K 31/403; A61K 31/455; A61K 31/495; A61K 31/496; A61K 31/519; A61K 31/5377; A61K 31/565; A61K 31/573; A61K 31/675; A61P 3/00

(57) ABSTRACT

Novel therapeutic uses of compounds for enhancing mitochondrial function and treating a mitochondrial disease are discovered by artificial intelligence (AI)-based in silico approaches. Methods of enhancing mitochondrial function and/or treating mitochondrial disease include administering an active compound to a subject in need. The active compound may be one or more compounds selected from the group consisting of preladenant, cinepazide, mitiglinide, deforolimus, mycophenolate-mofetil, megestrol, lercanidipine, fusidic acid, nimodipine, nicardipine, nitrendipine, benidipine, methazolamide, diethylcarbamazine, and pharmaceutically acceptable salts thereof.

### CROSS REFERENCE TO RELATED APPLICATIONS

This application claims the benefit of and priority to U.S. Provisional Application No. 63/057,992 filed Jul. 29, 2020, the content of which is incorporated herein by reference in its entirety.

### TECHNICAL FIELD

The present technology relates generally to novel therapeutic uses of compounds for enhancing mitochondrial function and treating mitochondrial diseases.

### BACKGROUND ART

### Background

Mitochondrion is an organelle present in most eukaryotic cells, of which primary function is oxidative phosphorylation, a process through which energy derived from metabolism of glucose or fatty acids is converted to adenosine triphosphate (ATP). ATP is then used to drive various energy-requiring biosynthetic reactions and other metabolic activities. In addition to generating cellular ATP, mitochondria are also involved in other cellular functions, such as cellular homeostasis, signaling pathways, and steroid synthesis.

Diseases arising from mitochondrial dysfunction may include for example, but not limited to, mitochondrial swelling due to mitochondrial membrane potential malfunction, functional diseases due to oxidative stress such as by the action of reactive oxygen species (ROS) or free radicals, functional diseases due to genetic mutations and diseases due to functional deficiency of oxidative phosphorylation mechanisms for energy production.

Mitochondria deteriorate with age, losing respiratory activity, accumulating damage to their DNA (mtDNA) and producing excessive amounts of ROS.

The mechanism of mitochondrial diseases (or pathological conditions associated with mitochondrial dysfunctions) has not been clearly understood until today. Recent evidence points to involvement of mitochondrial dysfunction in several diseases, for example, leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated 5 neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palmitoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-Arginine:Glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

Generally, treatment of a mitochondrial disease is palliative and aimed at treating symptoms and improving quality-of-life (QoL). Palliative treatments include, for example, administering vitamins, conserving energy, controlling dietary intake, and reducing stress on the body.

### Disclosure

### DESCRIPTION OF DRAWINGS

### Brief Description of Drawings

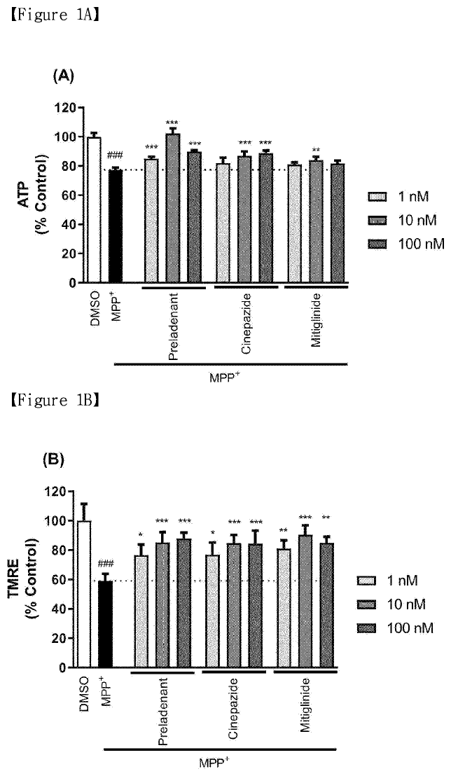

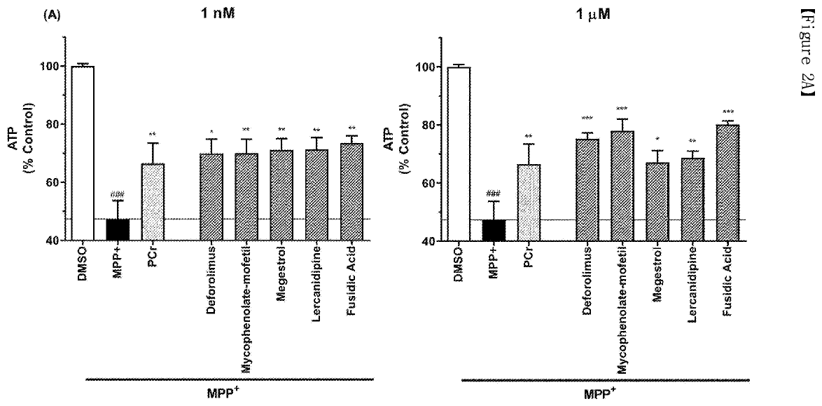

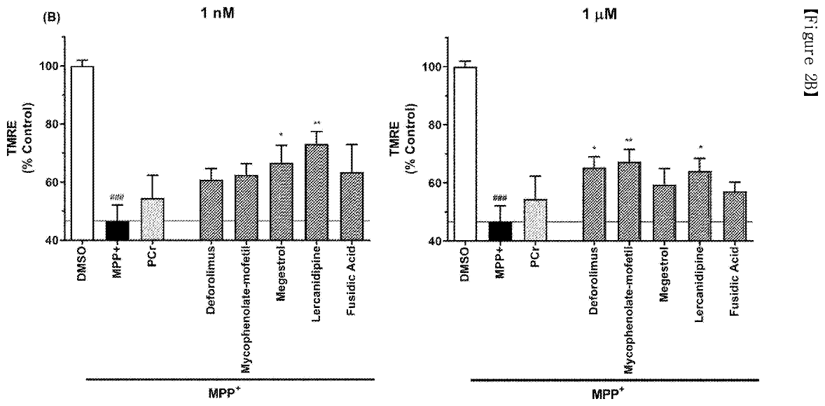

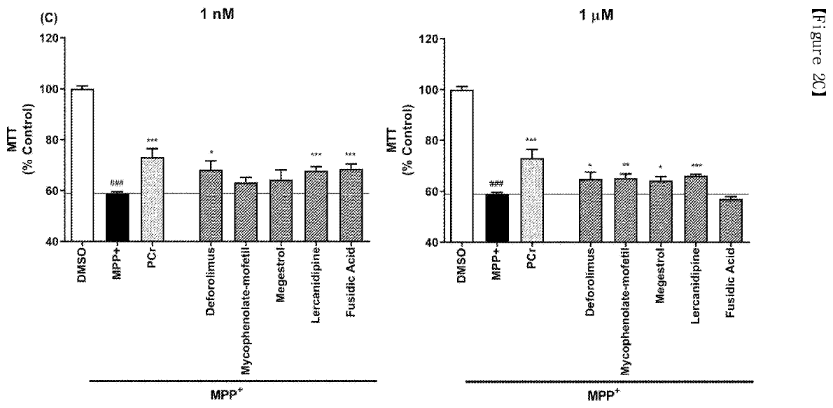

FIGS. 1(A) and 1(B) show protective effects of three drug compounds, preladenant, cinepazide and mitiglinide, that have drawn through transcriptional expression similarity-based approach. Quiescent SH-SY5Y cells were treated with each compound with indicated concentrations at 4 hours before 1 mM MPP+(1-methyl-4-phenylpyridinium; mitochondrial complex I inhibitor) treatment for 20 hours. FIG. 1(A) shows intracellular ATP content, and FIG. 1(B) shows tetramethylrhodamine ethylester (TMRE)-mediated mitochondrial membrane potential in a dose response manner. All values are reported as a percentage of the control (% Control). The data are plotted as the mean±standard error of measurement (n=4) (###p<0.001 vs. DMSO-control; *p<0.05, **p<0.01, and ***p<0.001 vs. MPP⁺-treated DMSO. p values are from a one-way ANOVA followed by Tukey's test).

FIGS. 2(A)-2(C) show protective effects of five drug compounds, deforolimus, mycophenolate-mofetil, megestrol, lercanidipine, and fusidic acid, that have drawn through structural similarity-based approach. Quiescent SH-SY5Y cells were treated with each compound with indicated concentrations at 4 hours before the treatment of 1 mM MPP⁺ for 20 hours. A reference drug, phosphocreatine (pCr) (1 mM) was also pretreated at 4 hours before the treatment of 1 mM MPP⁺ for 20 hours. FIG. 2(A) shows intracellular ATP content, FIG. 2(B) shows TMRE-mediated mitochondrial membrane potential, and FIG. 2(C) cell viability by methylthiazoletetrazolium (MIT) assay in a dose response manner. All values are reported as % Control. The data are plotted as the mean±standard error of measurement (n=6) (###p<0.001 vs. DMSO-control; *p<0.05, **p<0.01, and ***p<0.001 vs. MPP⁺-treated DMSO. p values are from a one-way ANOVA followed by Tukey's test).

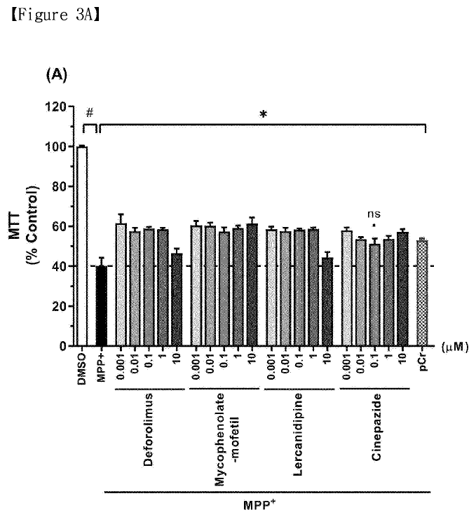

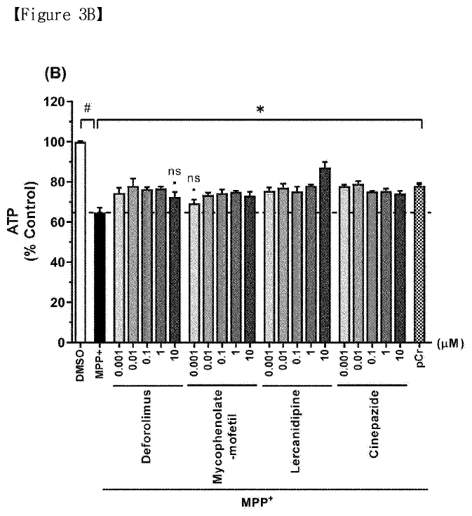

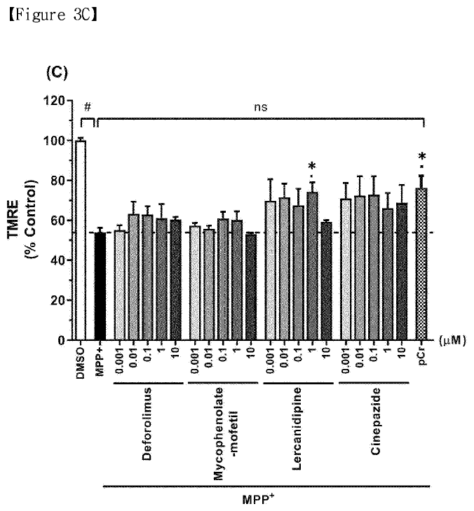

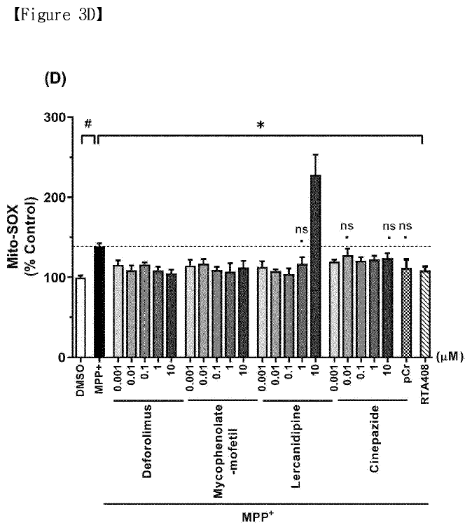

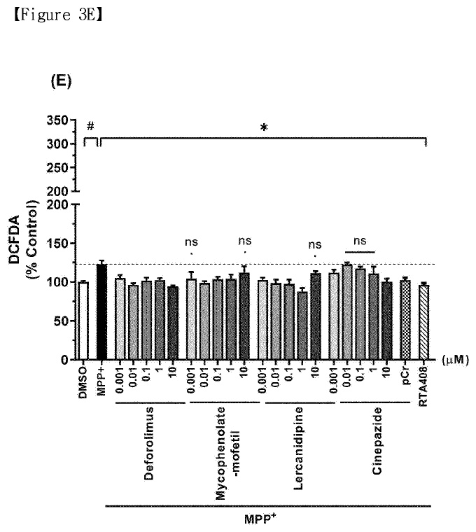

FIGS. 3(A)-3(E) show protective effects of four drug compounds, deforolimus, mycophenolate-mofetil, lercanidipine, and cinepazide, on mitochondrial functions. Quiescent SH-SY5Y cells were treated with each compound with indicated concentrations (0.001-10 UM) at 4 hours before the treatment of 1 mM MPP⁺ for 20 hours. A reference drug, phosphocreatine (pCr) (1 mM) was also pretreated at 4 hours before the treatment of 1 mM MPP⁺ for 20 hours. FIG. 3(A) shows cell viability by MIT assay, FIG. 3(B) shows intracellular ATP content, FIG. 3(C) shows TMRE-mediated mitochondrial membrane potential, FIG. 3(D) shows mitochondrial ROS content, and FIG. 3(E) shows intracellular ROS content in a dose response manner. All values are reported as % Control. The data are plotted as the mean±standard error of measurement (n=4) (#p<0.05 vs. DMSO-control; *p<0.05 vs. MPP⁺-treated DMSO. p values are from student's two-tailed t-test).

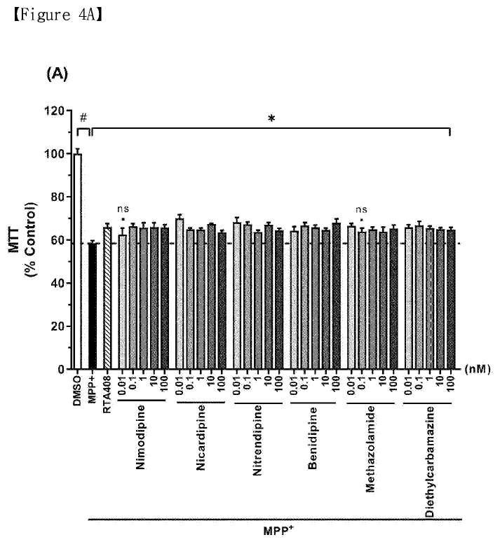

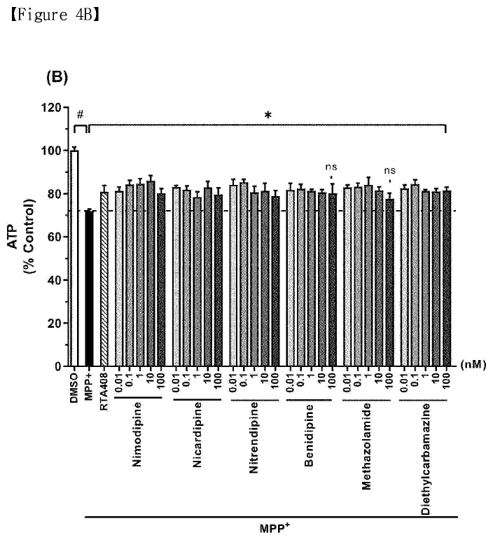

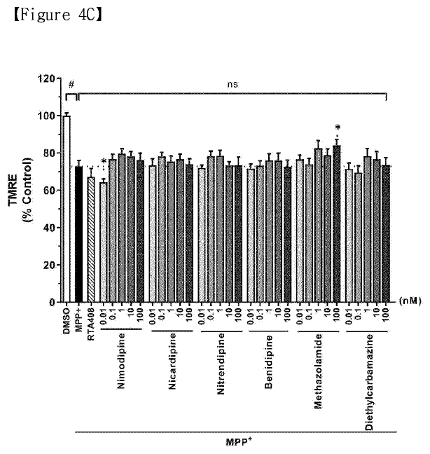

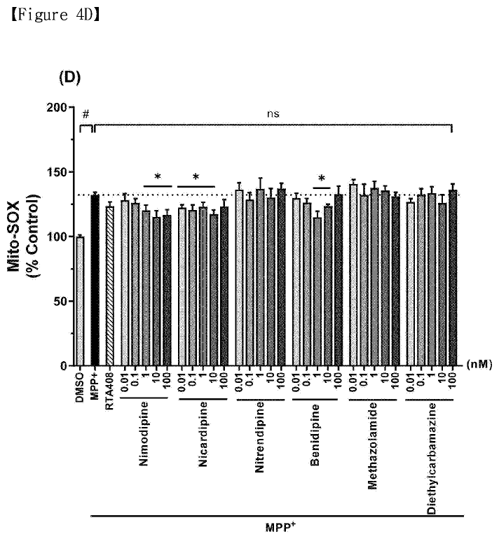

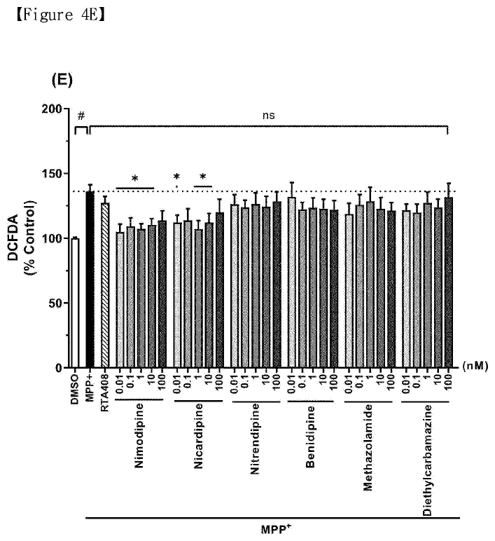

FIGS. 4(A)-4(E) show protective effects of six drug compounds, nimodipine, nicardipine, nitrendipine, benidipine, methazolamide, and diethylcarbamazine, that have drawn through structural similarity-based approach from seed compounds with mitochondrial mechanism of actions. Quiescent SH-SY5Y cells were treated with each compound with indicated concentrations (0.01-100 nM) at 4 hours before 1 mM MPP⁺ treatment for 20 hours. A reference drug, RTA408 (10 nM), was also pretreated at 4 hours before the treatment of 1 mM MPP⁺ for 20 hours. FIG. 4(A) shows cell viability by MTT assay, FIG. 4(B) shows intracellular ATP content, FIG. 4(C) shows TMRE-mediated mitochondrial membrane potential, FIG. 4(D) shows mitochondrial ROS content, and FIG. 4(E) shows intracellular ROS content in a dose response manner. All values are reported as % Control. The data are plotted as the mean±standard error of measurement (n=6) (#p<0.05 vs. DMSO-control; *p<0.05 vs. MPP⁺-treated control. p values are from student's two-tailed t-test).

### MODE FOR INVENTION

### Detailed Description

In order to discover novel therapeutic uses of existing drugs for mitochondrial diseases, different AI-based in silico approaches were applied to 4 million existing compounds. A group of compounds were selected as drug compounds to treat or ameliorate mitochondrial dysfunction by using two different approaches based on transcriptional and structural similarities of three different seed groups. The attributes of the seed groups including abilities for the increase of mitochondrial biogenesis, the treatment of primary mitochondrial diseases, and the advance of mitochondrial mechanisms, were found in the AI-drawn compounds. The protective abilities of selected compounds against mitochondrial defects or mitochondrial dysfunction were demonstrated empirically in human neuroblastic cells. All of the compounds reduced damages in mitochondrial membrane potential, oxidative stress, and ATP production as well as cell viability, and about a handful of compounds presented better recovery effect than phosphocreatine, the reference drug.

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. The headings provided herein are for convenience only and do not interpret the scope or meaning of the embodiments.

The present description in some embodiments provides a method for enhancing mitochondrial function in a subject which comprises administering to such subject an effective amount of one or more compounds selected from the group consisting of: preladenant, cinepazide, mitiglinide, deforolimus, mycophenolate-mofetil, megestrol, lercanidipine, fusidic acid, nimodipine, nicardipine, nitrendipine, benidipine, methazolamide, diethylcarbamazine, and a pharmaceutically acceptable salt, an isomer, a hydrate, and a tautomer thereof, preferably nimodipine, nicardipine, and a pharmaceutically acceptable salt, an isomer, a hydrate, and a tautomer thereof.

Pharmaceutically acceptable salts are those salts which can be administered as drugs or pharmaceuticals to humans and/or animals and which, upon administration, retain at least some of the biological activity of the free compound (neutral compound or non-salt compound). The desired salt of the listed compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic peptides with amino acids, such as aspartate salts and glutamate salts, can also be prepared. The desired salt of an acidic peptide can be prepared by methods known to those of skill in the art by treating the compound with a base. Examples of inorganic salts of acidic peptides include, but are not limited to, alkali metal and alkaline earth salts, such as sodium salts, potassium salts, magnesium salts, and calcium salts; ammonium salts; and aluminum salts. Examples of organic salts of acidic peptides include, but are not limited to, procaine, dibenzylamine, N-ethylpiperidine, N,N′-dibenzylethylenediamine, and triethylamine salts. Salts of acidic peptides with amino acids, such as lysine salts, can also be prepared.

The present description in some embodiments provides a method for enhancing mitochondrial function in a subject, wherein enhancing mitochondrial function is enhancing cell viabilities. The present description in some embodiments provides a method for enhancing mitochondrial function in a subject, wherein enhancing mitochondrial function is increasing intracellular ATP content. The present description in some embodiments provides a method for enhancing mitochondrial function in a subject, wherein enhancing mitochondrial function is increasing mitochondrial membrane potential (Δψm). The present description in some embodiments provides a method for enhancing mitochondrial function in a subject, wherein enhancing mitochondrial function is reducing mitochondrial ROS. The present description in some embodiments provides a method for enhancing mitochondrial function in a subject, wherein enhancing mitochondrial function is reducing intracellular ROS.

The present description in some embodiments provides a method for enhancing mitochondrial function in a subject, wherein enhancing mitochondrial function is, but not limited to, one or more events selected from the group consisting of: (i) enhancing cell viability; (ii) increasing intracellular ATP content; (iii) increasing mitochondrial membrane potential (Δψm); (iv) reducing mitochondrial ROS; and (v) reducing intracellular ROS, compared to prior to administering the active compound(s).

The present description in some embodiments provides a method for enhancing mitochondrial function in a subject, wherein the subject has one or more mitochondrial diseases. Particularly, the mitochondrial diseases comprise, but are not limited to, one or more selected from the group consisting of: leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palmitoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-Arginine:Glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

The present description in some embodiments provides a method for treating a mitochondrial disease by enhancing mitochondrial function in a subject, comprising administering to such subject an effective amount of one or more compounds selected from the group consisting of: preladenant, cinepazide, mitiglinide, deforolimus, mycophenolate-mofetil, megestrol, lercanidipine, fusidic acid, nimodipine, nicardipine, nitrendipine, benidipine, methazolamide, diethylcarbamazine, and a pharmaceutically acceptable salt, an isomer, a hydrate, and a tautomer thereof.

In some embodiments, the mitochondrial disease comprises, but is not limited to, one or more selected from the group consisting of: leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palmitoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-Arginine:Glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

### Definitions

The definitions of certain terms as used in this specification are provided below. Unless defined otherwise, all technical and scientific terms used herein generally have the same meaning as commonly understood by one of ordinary skill in the art to which this present technology belongs.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The term “biological sample” as used herein refers to a sample from the subject, and includes any bodily fluids, exudates, tissues or cells. Non-limiting examples include blood, plasma, serum, urine, tears, sputum, stool, saliva, nasal swabs, cells such as, but not limited to peripheral blood mononuclear cells (PBMCs), leukocytes, and tissue samples (e.g., biopsy samples). Samples can be fresh, frozen, or otherwise treated or preserved for evaluation by the methods disclosed herein.

As used herein the terms “enhancing” (or “enhance” or “enhancement”) is an approach for obtaining beneficial or desired results including clinical results. For example, enhancement can be of beneficial effect in a subject suffering from a disease or condition characterized by mitochondrial dysfunction, in that normalizing a biomarker of mitochondrial physiology may not achieve the optimum outcome for the subject; in such cases, enhancement of one or more biomarkers of mitochondrial physiology can be beneficial, for example, higher-than-normal levels of ATP, or lower-than normal levels of lactic acid (lactate) can be beneficial to such a subject.

As used herein, the terms “treating” (or “treat” or “treatment”) is an approach for obtaining beneficial or desired results including clinical results. For purposes of the present description, beneficial or desired clinical results include, but are not limited to, alleviating one or more symptoms of a disease, such as a mitochondrial disease, reducing one or more symptoms of a disease, such as a mitochondrial disease, preventing one or more symptoms of a disease, such as a mitochondrial disease, treating one or more symptoms of a disease, such as a mitochondrial disease, ameliorating one or more symptoms of a disease, such as a mitochondrial disease, delaying the onset of one or more symptoms of a disease, such as the onset of a mitochondrial disease, diminishing the extent of one or more symptoms of a disease, such as the extent of a mitochondrial disease, stabilizing a disease, such as a mitochondrial disease, delaying or slowing the progression of a disease, such as the progression of a mitochondrial disease, increasing the quality of life of the subjects suffering from a disease, such as the quality of life of the subject suffering from a mitochondrial disease, and/or prolonging survival of the subjects. In some embodiments, for example, a subject is successfully “treated” for a disease or disorder characterized by mitochondrial dysfunction if, after receiving a therapeutic amount of the active agents according to the methods described herein, the subject shows observable and/or measurable reduction in the disruption of mitochondrial oxidative phosphorylation. It is also to be appreciated that the various modes of treatment or prevention of medical diseases and conditions as described are intended to mean “substantial,” which includes total but also less than total treatment or prevention, and wherein some biologically or medically relevant result is achieved.

As used herein, the terms “effective amount” or “therapeutically effective amount” refer to an amount of a compound, composition or drug sufficient to enhance a specified function, such as enhance or increase desired and/or beneficial function, reduce or decrease undesired function; or to treat a specified disease, condition, or disease, such as ameliorate, palliate, lessen, and/or delay one or more of its symptoms. For example, in reference to a mitochondrial disease, an effective amount comprises an amount sufficient to delay development of a mitochondrial disease. In some embodiments, the effective amount is an amount sufficient to prevent or delay recurrence. An effective amount can be administered in one or more administrations. For example, in the case of mitochondrial diseases, the effective amount of the compound, composition or drug may: (i) inhibit, retard, slow to some extent and preferably stop muscular dysfunction; (ii) inhibit, retard, slow to some extent and preferably stop neurological dysfunction; (iii) inhibit, retard, slow to some extent respiratory dysfunction; (iv) inhibit, retard, slow to some extent morbidity; (v) prevent or delay occurrence and/or recurrence of a mitochondrial disease; and/or (vi) relieve to some extent one or more of the symptoms associated with having a mitochondrial disease.

As used herein, the “administration” of an agent, drug, or compound to a subject includes any route of introducing or delivering to a subject the agent, drug, or compound to perform its intended function. For the purposes of the present description, the administering is conducted, but not limited to, intravenously, intraportally, intra-arterially, intraperitoneally, intrahepatically, by hepatic arterial infusion, intravesicularly, subcutaneously, intrathecally, intrapulmonarily, intramuscularly, intratracheally, intraocularly, transdermally, intradermally, topically, orally, or by inhalation. Administration includes self-administration and the administration by another.

As used herein, the term “subject” (or “subjects”) may be a human or non-human subject, such as, not limited to, a non-human primate, a dog, a cat, a horse, a rodent, a rabbit, etc.

It is understood that aspects and embodiments of the present description described herein include “consisting” and/or “consisting essentially of” aspects and embodiments.

As used herein and in the appended claims, the singular forms “a,” “or,” and “the” include plural referents unless the context clearly dictates otherwise.

### Active Ingredient Compounds and Compositions

In embodiments, one or more selected from the group consisting of preladenant, cinepazide, mitiglinide, deforolimus, mycophenolate-mofetil, megestrol, lercanidipine, fusidic acid, nimodipine, nicardipine, nitrendipine, benidipine, methazolamide, diethylcarbamazine, and a pharmaceutically acceptable salt, an isomer, a hydrate, and a tautomer thereof, preferably nimodipine, nicardipine, and a pharmaceutically acceptable salt, an isomer, a hydrate, and a tautomer thereof are employed as an active ingredients.

Pharmaceutically acceptable salts are those salts which can be administered as drugs or pharmaceuticals to humans and/or animals and which, upon administration, retain at least some of the biological activity of the free compound (neutral compound or non-salt compound). The desired salt of the listed compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic peptides with amino acids, such as aspartate salts and glutamate salts, can also be prepared. The desired salt of an acidic peptide can be prepared by methods known to those of skill in the art by treating the compound with a base. Examples of inorganic salts of acidic peptides include, but are not limited to, alkali metal and alkaline earth salts, such as sodium salts, potassium salts, magnesium salts, zinc salts, copper salts, ferric salts, and calcium salts; ammonium salts; and aluminum salts. Examples of organic salts of acidic peptides include, but are not limited to, procaine, dibenzylamine, N-ethylpiperidine, N,N′-dibenzylethylenedizmine and triethylamine salts. Salts of acidic peptides with amino acids, such as lysine salts, can also be prepared.

When two or more active ingredients are employed, the active ingredients may be separately, sequentially, or simultaneously. As used herein, the term “separately” refers to an administration of at least two active ingredients at the same time or at substantially the same time by different routes. As used herein, the term “sequentially” refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. As used herein, the term “simultaneously” refers to the administration of at least two active ingredients by the same route and at the same time or at substantially the same time.

The active agents described herein can be incorporated into pharmaceutical compositions for administration, singly or in combination, to a subject for the treatment or prevention of a medical disease or condition described herein, such as mitochondrial dysfunction associated conditions and symptoms. Such compositions typically include the active agent (e.g., preladenant, cinepazide, mitiglinide, deforolimus, mycophenolate-mofetil, megestrol, lercanidipine, fusidic acid, nimodipine, nicardipine, nitrendipine, benidipine, methazolamide, diethylcarbamazine, and a pharmaceutically acceptable salt, an isomer, a hydrate, and a tautomer thereof) and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., treatment for one week).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor, or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The composition can include a carrier, which can be a solvent or dispersion medium containing, for example, water, ethanol, polyol (for example, glycerol, propylene glycol, liquid polyethylene glycol, and the like), and suitable mixtures thereof.

The composition may include various antibacterial and antifungal agents, for example, parabens, chlorobutanol, phenol, ascorbic acid, thiomerasol, and the like. Glutathione and other antioxidants can be included to prevent oxidation. The composition also may include isotonic agents such as, for example, sugars, polyalcohols such as mannitol, sorbitol, or sodium chloride in the composition. Injectable compositions may include an agent that delays absorption, for example, aluminum monostearate or gelatin, to prolong absorption.

The composition may be in a sterile injectable solution that can be prepared by incorporating the active ingredient compound in the required amount in an appropriate solvent with one or a combination of ingredients enumerated above, as required, followed by filtered sterilization. For example, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose, trehalose, or saccharin; or a flavoring agent such as peppermint, methyl salicylate, or citrus (orange) or other fruit (grapefruit, grape, apple, pineapple, and the like) flavoring agent.

For administration by inhalation, the active agent such as an aromatic-cationic peptide of the present technology can be delivered in the form of an aerosol spray from a pressurized container or dispenser, which contains a suitable propellant, e.g., a gas such as carbon dioxide, or a nebulizer.

Systemic administration of an active agent such as an aromatic-cationic peptide of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

The active compound can be formulated in a carrier system. The carrier can be a colloidal system. The colloidal system can be a liposome, a phospholipid bilayer vehicle. In one embodiment, the therapeutic agent such as an aromatic-cationic peptide is encapsulated in a liposome while maintaining peptide integrity. One skilled in the art would appreciate that there are a variety of methods to prepare liposomes. Liposomal formulations can delay clearance and increase cellular uptake. See e.g., Reddy, Ann. Pharmacother., 34(7-8): 915-923 (2000)). The active compound can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

Examples of polymer microsphere sustained release formulations are described in, for example, PCT publication WO 99/15154 (Tracy, et al.), U.S. Pat. Nos. 5,674,534 and 5,716,644 (both to Zale, et al.).

In some embodiments, the active compounds may be prepared with carriers that will protect the active compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques.

Typically, an effective amount of the active compound, sufficient for achieving a therapeutic or prophylactic effect, ranges from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. In one embodiment, a single dosage of an active compound ranges from 0.001-10,000 micrograms per kg body weight.

These and other aspects and advantages of the present invention will become apparent from the subsequent detailed description and the appended claims. It is to be understood that one, or all of the properties of the various embodiments described herein may be combined to form other embodiments of the present description.

### EXAMPLE

The presently disclosed subject matter will be better understood by reference to the following Example, which is provided as exemplary of the presently disclosed subject matter, and not by way of limitation.

### Example 1: AI-Aided Discovery of Repurposed Drug Compounds for Mitochondrial Diseases

### Method

In order to screen compounds that are suitable for enhancing mitochondrial function and/or treating mitochondrial disease(s), a set of AI-based in silico approaches were applied to 4 million existing compounds. In silico-based approaches to screen active compounds for mitochondrial diseases, which were applied to the known compounds, are below.
Module 1—Graphic knowledge database
Module 2—Drug repurposing model based on the drug-perturbed transcriptional expression pattern of genes
Module 3—Clustering model of the pathophysiological property-embodied latent space of Module 2
Module 4—Drug repurposing model based on structural and drug-like similarity
Module 5—Drug-focused information searching model

### The 1st Seed Group

In order to be applied to AI-based drug repurposing models as seed compounds, three therapeutic compounds, bezafibrate, curcumin, and resveratrol, which are known to increase mitochondrial biogenesis were selected from literatures (Hirano M et al., Emerging therapies for mitochondrial diseases. Essays Biochem. 2018; 62(3):467-481; El-Hattab A W et al., Therapies for mitochondrial diseases and current clinical trials. Mol Genet Metab. 2017; 122(3): 1-9; Nightingale H. et al., Emerging therapies for mitochondrial diseases. Brain. 2016; 139(6): 1633-1648).

Further, additional four drugs, acipimox, pioglitazone, rosiglitazone, and varenicline, were selected by Module I, and added to the 1^{st} seed group in order to enhance the property for mitochondrial biogenesis in the 1^{st} seed group. The four drugs that Module 1 suggested have similar connecting properties with the above three drugs (bezafibrate, curcumin, and resveratrol) in terms of drug-targets, drug-pathways, and disease-pathways.

The association of these compounds to the mitochondrial pathophysiology was proved by literature; acipimox showed a beneficial effect on muscle mitochondrial function as an NAD⁺ precursor in clinical study (van de Weijer T et al., Evidence for a direct effect of the NAD⁺ precursor Acipimox on muscle mitochondrial function in humans. Diabetes. 2015; 64(4): 1193-1201) and entered a new clinical study (NCT03325491) for muscle performance with impaired mitochondrial function after the positive result of previous clinical study with muscle performance in aged men (NCT02792621). Pioglitazone and rosiglitazone are reported to induce mitochondrial biogenesis as a peroxi some proliferator-activated receptor (PPAR) gamma agonist in adipose tissue and patient cells with Friedreich's ataxia and Down syndrome (Bogacka I. et al., Pioglitazone Induces Mitochondrial Biogenesis in Human Subcutaneous Adipose Tissue In Vivo. Diabetes. 2005; 54(5): 1392-1399; Aranca T V. et al., Emerging therapies in Friedreich's ataxia. Neurodegener Dis Manag. 2016; 6(1):49-65; Mollo N. et al., Pioglitazone Improves Mitochondrial Organization and Bioenergetics in Down Syndrome Cells. Front Genet. 2019; 10). Varenicline is a partial agonist of the alpha 4/beta 2 subtype of the nicotinic acetylcholine receptor. Clinical trials of varenicline (NCT00803868) and pioglitazone (NCT00811681) have conducted for Friedreich's ataxia, a neurodegenerative movement disease with a genetic defect in FXN whose encoded protein has an essential role for mitochondrial respiratory complexes. Therefore, these four therapeutic compounds (acipimox, pioglitazone, rosiglitazone, and varenicline) were designated to a mitochondrial biogenesis seed group together with three known therapeutic compounds mentioned above (bezafibrate, curcumin, and resveratrol).

### The 2nd Seed Group and the 3rd Seed Group

The therapeutic molecules found in reported clinical trials for mitochondrial diseases were collected, and 13 molecules were selected as the second seed group (TABLE 1).

TABLE 1

| Seed | Mechanism | Reference |
|---|---|---|
| CoQ10 | OXPHOS/ROS | NCT00432744 |
| EPI-743 | Antioxidant | NCT01370447 |
| Idebenone | Antioxidant | NCT00887562 |
| L-Arginine | NO precursor | NCT01603446 |
| Lipoic acid | Antioxidant | NCT02348125 |
| Bezafibrate | Mitochondrial biogenesis | NCT02398201 |
| Curcumin | Antioxidant, | NCT00528151 |
| | Mitochondrial biogenesis | |
| Cyclosporine A | Inhibition of | NCT02176733; |
| | mitochondrial PTP | NCT02616484 |
| RP103 | Cystine-depleting agent | NCT02023866; |
| | | NCT02473445 |
| Omaveloxolone | Antioxidant, | NCT0225542 |
| | Nrf2 Activator, | |
| | NFkb Inhibitor | |
| KH176 | Antioxidant | NCT02909400; |
| | | NCT02544217 |
| KL1333 | NAD+ modulator | NCT03056209 |
| Pyruvate | NAD donor | JMA-IIA00093 |

This seed group of the reported clinical trial drugs consisted of antioxidants, mitochondrial biogenesis boosters, NAD⁺ modulators, and so on. Antioxidant and oxidative stress-modulating compounds were the most dominant.

To supplement recent pathological, physiological, and pharmaceutical advances in mitochondrial biology, the third seed group was prepared additionally. Drug compounds in reported preclinical and clinical trials for neurodegenerative diseases, oxidative stress-induced diseases, or metabolic diseases were applied to Module 1 to identify compounds with the mitochondrial mechanism of action (MOA). The diseases were used for Module 1 are including but not limited to Parkinson's disease, Alzheimer's disease, amyotrophic lateral sclerosis, multiple sclerosis, ischemia-reperfusion injury, NASH, cardiovascular diseases, chronic obstructive pulmonary disease, chronic kidney disease, and diabetes. In addition, the therapeutic molecules designed for emerging targets for mitochondrial diseases were interrogated to Module 1. The emerging targets noted for therapy of mitochondrial diseases included, but were not limited to, DRP1 inhibitors, 5-1 ipoxygenase inhibitors, SIRT1 activators, PARP-1 inhibitors, and NRF2 activators. As a result, among therapeutic molecules suggested by Module 1, ten compounds whose mitochondrial MOAs were proved in in vivo models were chosen as a mitochondrial MOA seed group (the 3rd seed group). They are imeglimin (Vial G. et al., Imeglimin Normalizes Glucose Tolerance and Insulin Sensitivity and Improves Mitochondrial Function in Liver of a High-Fat, High-Sucrose Diet Mice Model. Diabetes. 2015; 64(6):2254-2264), blarcamesine (Missling C U, ANAVEX 2-73, a Clinical Candidate for Neurodevelopmental Disorders: New data Including AnC-Seizure data in Angelman Syndrome.: 24), isradipine (Guzman J N, et al., Systemic isradipine treatment diminishes calcium-dependent mitochondrial oxidant stress. J Clin Invest. 2018; 128(6): 2266-2280), latrepirdine (Eckert S H, et al., Mitochondrial Pharmacology of Dimebon (Latrepirdine) Calls for a New Look at its Possible Therapeutic Potential in Alzheimer's Disease. Aging Dis. 2018; 9(4): 729-744), resveratrol (Decui L, et al., Micronized resveratrol shows promising effects in a seizure model in zebrafish and signalizes an important advance in epilepsy treatment. Epilepsy Res. 2020; 159: 106243), nicotinamide (Bayrakdar E T, et al., Ex vivo protective effects of nicotinamide and 3-aminobenzamide on rat synaptosomes treated with Aβ(1-42). Cell Biochem Funct. 2014; 32(7):557-564), Mdivi-1 (Bido S, et al., Mitochondrial division inhibitor-1 is neuroprotective in the A53T-α-synuclein rat model of Parkinson's disease. Sci Rep. 2017; 7), CNB-001 (Jayaraj R L, et al., CNB-001 a novel curcumin derivative, guards dopamine neurons in MPTP model of Parkinson's disease. Biomed Res Int. 2014; 2014:236182), CPUY192018 (Lu M-C, et al., CPUY192018, a potent inhibitor of the Keap1-Nrf2 protein-protein interaction, alleviates renal inflammation in mice by restricting oxidative stress and NF-κB activation. Redox Biol. 2019; 26; Lu M-C, et al., An inhibitor of the Keap1-Nrf2 protein-protein interaction protects NCM460 colonic cells and alleviates experimental colitis. Sci Rep. 2016; 6), and compound 14 (Ma B, et al., Design, synthesis and identification of novel, orally bioavailable non-covalent Nrf2 activators. Bioorganic & Medicinal Chemistry Letters. Published online Dec. 2, 2019: 126852).

### Assessment of Candidate Drug Compounds for Mitochondrial Diseases Treatment Efficacy by Using Drug Perturbed Transcriptional Similarity

The mitochondrial biogenesis seed group (the 1st seed group) was applied to Module 2 and Module 3. Module 2 is a drug repurposing model based on transcriptional similarity by training drug-perturbed gene expression datasets obtained from Broad Institute LINCS project (Subramanian A., et al., A Next Generation Connectivity Map: L1000 Platform And The First 1,000,000 Profiles. bioRxiv. Published online May 10, 2017: 136168; Musa A., et al., L1000 Viewer: A Search Engine and Web Interface for the LINCS Data Repository. Front Genet. 2019; 10). It predicts new uses of more than 11,000 drugs in the LINCS library. Module 3 was created because the mitochondrial diseases were not classified in the drug indication system of Module 2. In Module 3, each given vector of the drug perturbed gene expression data in the latent space of Module 2 under optimized hyper-parameters was trained and clustered. The distribution pattern and the frequency of the gene expression data of a drug in the clusters were reflected in our scoring system. As a consequence, so-called neighbor compounds were drawn from Module 3, by using drug-perturbed transcriptional similarity of a seed compound. Indeed, a drug-target similarity was validated in silico between seed compounds and neighbor compounds when anticancer drugs in a cancer validation set were applied to Module 3.

### Assessment of Candidate Drug Compounds for Mitochondrial Diseases Treatment Efficacy by Using Structural and Drug-Like Similarity

Module 4 was constructed by using a dataset of about 4 million SMILES strings with physical activities from public databases. The deep learning algorithm of Module 4 created a latent chemical space in which structural and drug-like similarity of compounds is embodied, and it is allowed discovering neighbor compounds with desired properties from a seed compound.

The clinical trial seed group (the 2^{nd} seed group) and the mitochondrial MOA seed group (the 3^{rd} seed group) were applied to Module 4. The drug compounds from the clinical trial seed group were selected by calculation of cosine distance and the drug compounds list from the mitochondrial MOA seed group was drawn by computing both cosine distance and Euclidean distance. The neighbor compounds of each seed were identified by mapping their SMILES with CAS registry numbers. The compounds with no CAS registry number were eliminated from the lists.

### Filtering

All the neighbor compounds were applied to Module 5, which enabled researchers to search drug-focused information in scientific literature and patents. Module 5 removed the compounds that were reported as therapeutics on the primary mitochondrial diseases attributed to dysfunctions in mitochondrial respiratory chain reaction, demonstrated to have mitochondrial toxicity by empirical tests, and reported to be toxic to patients with mitochondrial diseases or to mitochondrial function and/or mitochondrial homeostasis including, but not limited to the cellular events such as apoptosis, ROS production, and inhibition of mitochondrial respiratory complexes. Additionally, compounds with a toxicity warning or developmental disadvantages including preclinical, parenteral, commercially not available, and having sever side effect were filtered out.

### Results

A list of neighbor compounds was drawn through STANDIGM AI-based drug repurposing process by using 27 seed compounds for their therapeutic and/or mechanistic activities for mitochondrial diseases or mitochondria-related diseases.

After the filtering process, three drug compounds (i.e., preladenant, cinepazide, and mitiglinide) were selected through the drug-perturbed transcriptional similarity approach, five drug compounds (i.e., deforolimus, mycophenolate-mofetil, megestrol, lercanidipine, and fusidic acid) were selected through the approach of using structural similarity with the clinical trial seed group, and six compounds (i.e., nimodipine, nicardipine, nitrendipine, benidipine, methazolamide, and diethylcarbamazine) were selected through the approach of using structural similarity with the mitochondrial MOA seed group.

Table 2 shows fourteen (14) compounds selected from each approach with its IUPAC name and chemical structure. In Example 2 below, some of 14 compounds were tested as salt forms, and their salt forms are also described in Table 2. However, the pharmaceutically acceptable salts of fourteen (14) compounds are not limited to the salt forms described in Table 2.

TABLE 2

| | IUPAC | |
|---|---|---|
| Name | name | Structure |
| pre [symbol] | [symbol] | 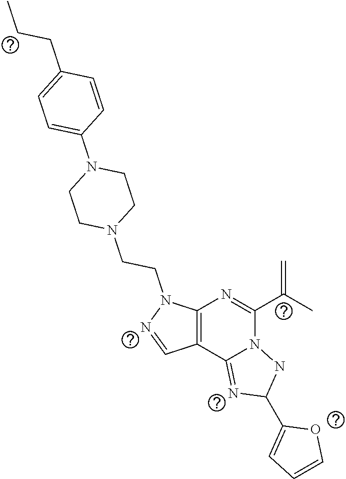 [SMILES: C=C(C)C1=Nc2c(cnn2CCN2CCN(c3ccc(CCC)cc3)CC2)C2=NC(c3ccco3)NN12] |
| cinepazide | [symbol] | 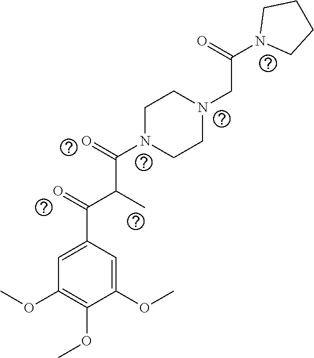 [SMILES: COc1cc(C(=O)C(C)C(=O)N2CCN(CC(=O)N3CCCC3)CC2)cc(OC)c1OC] |
| mitiglinide | [symbol] | 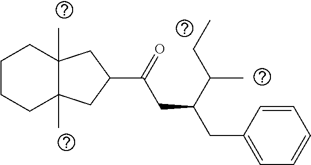 [SMILES: CCC(C)[C@H](CC(=O)C1CC2(C)CCCCC2(C)C1)Cc1ccccc1] |
| de [symbol] | [symbol] | 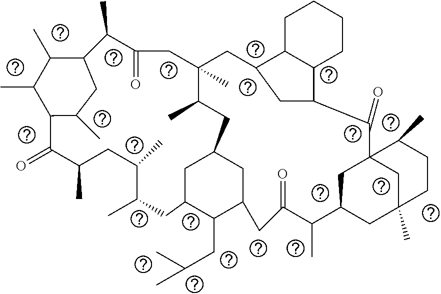 [SMILES: CC(C)CC1C2CC(=O)C(C)[C@H]3CC4(C[C@@](C)(CC[C@@H]4C)C3)C(=O)C3CC(C[C@]4(C)CC(=O)[C@H](C)C5CC(C)C(C(=O)[C@H](C)C[C@H](C)[C@@H](C)CC1C[C@H](C2)C[C@H]4C)C(C)C5C)C1CCCCC31] |
| mycophenolate- [symbol] | 2-morpholin-4-ylethyl (E)-6-(4-hydroxy- [symbol] - methoxy-7-methyl-3- oxo-1H-2-benzofuran-5- yl)-4-methylhex-4- enoate | 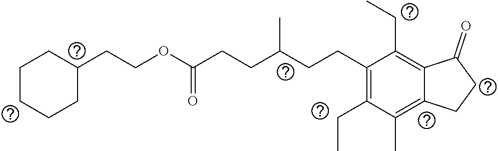 [SMILES: CCc1c(C)c2c(c(CC)c1CCC(C)CCC(=O)OCCC1CCCCC1)C(=O)CC2] |
| megestrol | (8R,9S,10R,13S,14S, 17R)-17-acetyl-17- hydroxy-6,10, 13-trimethyl- 2,8,9,11,12,14,15, 16-octahydro-1H- cyclopenta[a] phenanthren-3-one | 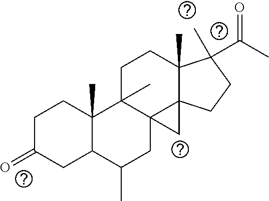 |
| [symbol] | 5-O-[1-[3,3- diphenylpropyl (methyl)amino]-2- methylpropan-2-yl] 3-O-methyl 2,6- dimethyl-4- (3-nitrophenyl)-1,4- dihydropyridine-3, 5-dicarboxylate | 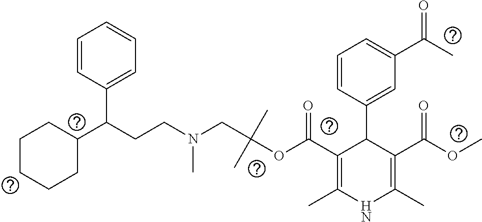 [SMILES: COC(=O)C1=C(C)NC(C)=C(C(=O)OC(C)(C)CN(C)CCC(c2ccccc2)C2CCCCC2)C1c1cccc(C(C)=O)c1] |
| [symbol] usidic acid | (2Z)-2-[(3R,4S,5S, 8S,9S,10S,11R, 13R,14S,16S)- 16-acetyloxy-3,11- dihydroxy-4, [symbol] ,10, 14-tetramethyl- 2,3,4,5,6,7,9,11,12, 13,15,16-dodeca- hydro-1H-cyclopenta [a]phenanthren-17-yli- dene]-6-methylhept- 5-enoic acid | 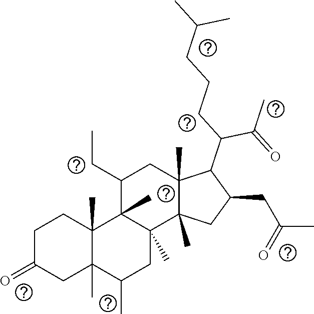 |
| nimodipine | 3-O-(2-methoxyethyl) 5-O-propoan-2-yl 2,6-dimethyl-4-(3- nitrophenyl)-1,4- dihydropyridine-3.5- dicarboxylate | 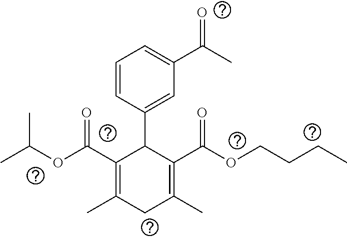 [SMILES: CCCCOC(=O)C1=C(C)CC(C)=C(C(=O)OC(C)C)C1c1cccc(C(C)=O)c1] |
| nicardipine | 5-O-[2-[benzy l(methyl)amino] ethy [symbol] | 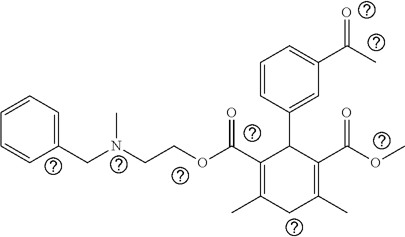 [SMILES: COC(=O)C1=C(C)CC(C)=C(C(=O)OCCN(C)Cc2ccccc2)C1c1cccc(C(C)=O)c1] |
| nitrendipine | 5-O-ethyl 3-O-methyl 2,6-dimethyl-4-(3- nitrophenyl)-1,4- didropyridine-3,5- dicarboxylate | 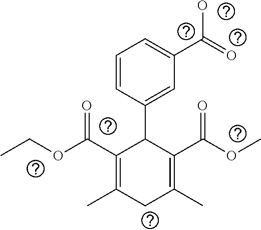 [SMILES: CCOC(=O)C1=C(C)CC(C)=C(C(=O)OC)C1c1cccc(C(=O)O)c1] |
| benidipine | 5-O[(3R)-1- benzylpiperidin-3- yl] 3-O-methyl (4R)-2,6-dimethyl-4- (3-nitrophenyl)-1,4- dihydropyridine-3, [symbol] dicarboxylate | 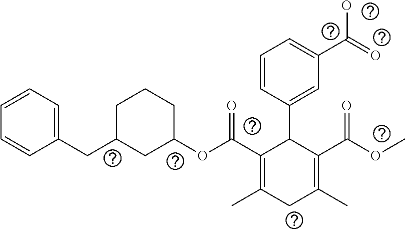 [SMILES: COC(=O)C1=C(C)CC(C)=C(C(=O)OC2CCCC(Cc3ccccc3)C2)C1c1cccc(C(=O)O)c1] |
| methazolamide | N-(3-methyl-5- sulfamoyl-1,3,4- thiadazol-2- ylidene)acetamide | 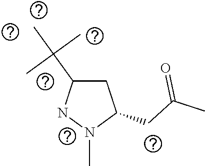 |
| diethylcarbamazine | N,N-diethyl-4- methylpiperazine- 1-carboxamide | 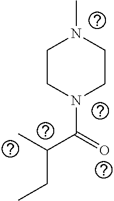 |

| | Compound | | |
|---|---|---|---|
| | for | IUPAC | |
| Name | experiments | name | Structure |
| preladenant | | | |
| cinepazide | cinepazide mal [symbol] | (Z)-but-2- enectoic acid;(E)- 1-[4-2-oxo-2- pyrrolidin-1- ylethyl)piperazin- 1-yl]-3-(3,4,5- trimethoxyphenyl)prop-2-en-1-one | 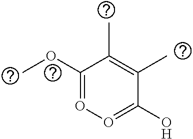 |
| | | | 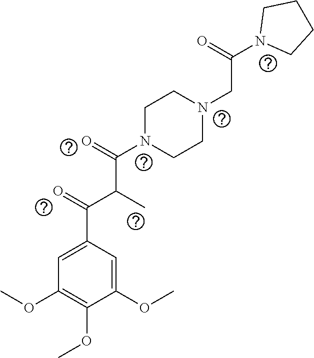 [SMILES: COc1cc(C(=O)C(C)C(=O)N2CCN(CC(=O)N3CCCC3)CC2)cc(OC)c1OC] |
| mitiglinide | mitiglinide calcium hydrate | calcium;(2S)- 4-[(3aS, 7aR)-1,3,3a ,4,5,6,7,7a- octahdro- isoindol-2-yl]- 2-benzyl-4- oxobutanoate | 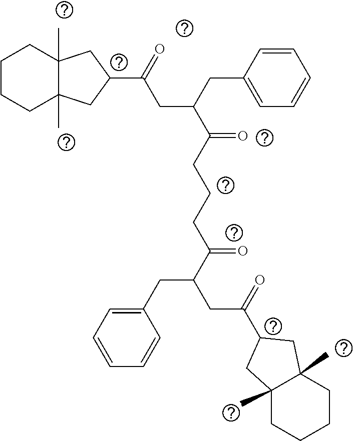 [SMILES: CC12CCCCC1(C)CC(C(=O)CC(Cc1ccccc1)C(=O)CCCC(=O)C(CC(=O)C1C[C@]3(C)CCCC[C@]3(C)C1)Cc1ccccc1)C2] |
| deflorolimus | | | |
| mycoph [symbol] | | | |
| megestrol | megestrol Acetate | [symbol] | 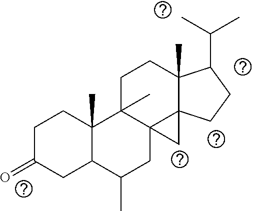 |
| [symbol] | larcan- [symbol] hydrochloride | [symbol] | 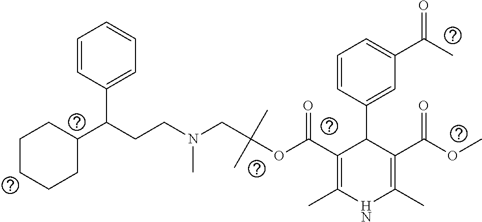 [SMILES: COC(=O)C1=C(C)NC(C)=C(C(=O)OC(C)(C)CN(C)CCC(c2ccccc2)C2CCCCC2)C1c1cccc(C(C)=O)c1] |
| [symbol] usidic acid | [symbol] sodium | [symbol] | 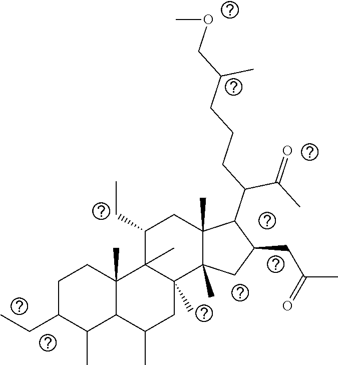 |
| nimodipine | | | |
| nicardipine | nicardipine hydrochloride | 5-O-[2- [benzyl (methyl) amino]ethyl] 3-O-methyl 2.6-dimethyl- 4-(3- nitrophenyl)- 1,4- dihydro- pyridine- 3,5- dicarboxylate; hydrochloride | 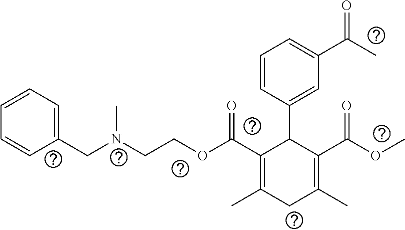 [SMILES: COC(=O)C1=C(C)CC(C)=C(C(=O)OCCN(C)Cc2ccccc2)C1c1cccc(C(C)=O)c1] |
| nitrendipine | | | |
| benidipine | benidipine hydrochloride | 5-O-[(3R)-1- benzylpiperidin- 3-yl] 3-O-methyl (4R)-2,6- dimethyl-4-(3- mitrophenyl)-1,4- dihydropyridine- 3,5-dicarboxylate | 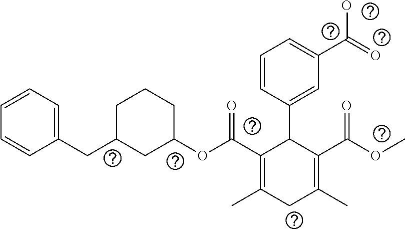 [SMILES: COC(=O)C1=C(C)CC(C)=C(C(=O)OC2CCCC(Cc3ccccc3)C2)C1c1cccc(C(=O)O)c1] |
| methazol- amide diethyl- carbamazine | diethyl- carbamazine | N,N- diethyl-4- methyl- piperazine- 1-carboximide, 2-hydroxy- propane-1,2, 3- tricarboxylic acid | 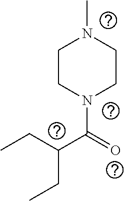 |
| | | | 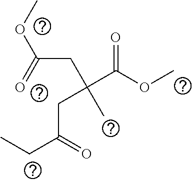 [SMILES: CCC(=O)CC(C)(CC(=O)OC)C(=O)OC] |

[symbol]

indicates data missing or illegible when filed

### Example 2: In Vitro Validation of AI-Discovered Drug Compounds for Mitochondrial Diseases Treatment Efficacy

### Method

### Cell Culture and Chemical Treatment

SH-SY5Y human neuroblastoma cells were cultured in Dulbecco's Modified Eagle Medium (DMEM)/F12 supplemented with 10% fetal bovine serum (FBS), 100 U/ml penicillin, and 100 μg/ml streptomycin (complete media, CM) at 37° C./5% CO₂. Cells seeded at 5×10⁴ cells/well were cultured in 96-well plates for 24 hours followed by incubation in serum-deficient media (SDM, DMEM/F12 containing 0.5% FBS) for 16 hours. Cells in SDM were pre-treated with each chemical for 4 hours, followed by incubation with 1 mM mitochondrial complex I inhibitor (MPP⁺) or dimethyl sulfoxide (DMSO) vehicle for 20 hours.

### Cell Viability Assay—Methylthiazoletetrazolium (MTT) Assay

MTT assay is a measure of mitochondrial dehydrogenase activity in live cells. MTT assay examines viable cell amount by measuring the mitochondrial metabolic rate (Rai Y. et al., Mitochondrial biogenesis and metabolic hyperactivation limits the application of MIT assay in the estimation of radiation induced growth inhibition, Scientific Reports. 2018; 8(1): 1531), presenting the metabolic viability of cells. SH-SY5Y cells (2.5×10⁴ cells/well) in SDM in 96-well plates were treated with reagents as indicated and further incubated with 3-(4,5 dimethylthiazol-2-yl)-2,5-diphenyl-tetrazolium bromide (MTT, Sigma) solution (0.2 mg/ml MIT in PBS) for 4 hours. The MTT formazan precipitates formed by live cells were dissolved in 100 μl of 0.04 N HCl/isopropanol. The absorbance at 540 nm was measured by ELISA microplate reader (Molecular Devices, Sunnyvale, CA).

### Intracellular ATP Assay—Luciferase-Based Assay

Intracellular ATP content was measured by luciferin-luciferase reaction with Cell Titer luciferase kit (Promega) according to the manufacturer's instructions. 20 μl cell lysates were mixed with 20 μl of the luciferin-luciferase reaction buffer and incubated at 20° C. for 10 min. The luminescence signal was measured with an LB 9501 LUMAT® luminometer (Berthold, German). It was subtracted (i.e., corrected) from the signal that a background luminescence value in control wells containing medium without cells. The amounts of ATP content were normalized to protein concentration. All data were presented as a percent of control after calculation.

### Mitochondrial Membrane Potential (Δψm) Assay and Reactive Oxygen Species (ROS) Assay

Mitochondrial membrane potential was measured using tetramethylrhodamine ethylester (TMRE, Molecular Probe). The cells in black 96-well culture plate were incubated with 200 nM TMRE and HOECHST® 33342 (0.5 μM) for 30 min at 37° C. in phenol red-free SDM.

Similarly, total and mitochondrial ROS generations were measured using CM-H2DCFDA and MitoSox, respectively. Cells were incubated with 1 UM CM-H2DCFDA or 5 μM MitoSox and 0.5 μM Hoechst 33342 for 1 hour at 37° C.

Fluorescence intensities at 550 nm/580 nm for TMRE, at 510 nm/580 nm for MitoSox, or at 494 nm/522 nm for CM-H2DCFDA were normalized by HOECHST® intensity at 355 nm/480 nm (Spectramax Gemini EM, Molecular Devices, Sunnyvale, CA, USA).

### Results

Three (3) drug compounds (preladenant, cinepazide, and mitiglinide) showed protective effects

Quiescent human neuroblastoma cells (SH-SY5Y) cells were treated with each drug compounds at various concentrations (i.e., 1 nM, 1 UM) for 4 hours before cells were incubated with 1 mM MPP⁺ for 20 hours.

As shown in FIGS. 1(A) and 1(B), the MPP⁺ induced cellular and mitochondrial damages in human neuroblastoma cells (SH-SY5Y) were reduced by all three compounds from the approach of the transcriptional expression similarity. The three compounds increase intracellular ATP content and TMRE-mediated mitochondrial membrane potential in a dose-dependent manner (1 nM, 10 nM, and 100 nM), showing their beneficial ability for the increase of mitochondrial activity.

### Five (5) Drug Compounds (Deforolimus, Mycophenolate-Mofetil, Megestrol, Lercanidipine, and Fusidic Acid) Showed Protective Effects

Five drug compounds, deforolimus, mycophenolate-mofetil, megestrol, lercanidipine, and fusidic acid, which were drawn through the structural similarity-based approach, were tested to validate their protective effect in a dose-dependent manner.

As shown in FIGS. 2(A) and 2(B), all of the drug compounds showed their protective effects against MPP⁺ in intracellular ATP content and TMRE-mediated mitochondrial membrane potential at both concentrations of 1 nM and 1 μM. Interestingly, all of the drug compounds presented better recovering ability than phosphocreatine, the control and therapeutic compound for mitochondrial diseases in clinical trials.

As shown in FIG. 2(C), the metabolic cell viability increased when the drug compounds were treated prior to MPP⁺. Most of the drug compounds rescued the cells from MPP⁺-induced death with statistical significance.

### Further Validation of Protective Effects

Four drug compounds (i.e., deforolimus, mycophenolate-mofetil, lercanidipine, and cinepazide) were tested to evaluate their protective effects and dose-dependent effects.

As shown in FIGS. 3(A) and 3(B), the drug compounds showed beneficial efficacy in cell viability and intracellular ATP content through the concentration of 1 nM to 10 μM with a similar level of efficacy 1 μM of phosphocreatine presented. As shown in FIG. 3(C), the protective effects of the drug compounds on TMRE-mediated mitochondrial mem-brane potential were observed through the concentration of 1 nM to 10 μM.

As shown in FIGS. 3(D) and 3(E), the MPP⁺-induced production of mitochondrial and intracellular ROS were reduced by the drug compounds. The levels of reduction were similar to phosphocreatine or RTA408, a promising therapeutic compound that improves the oxidative stress condition in mitochondria.

Six (6) Drug Compounds Drawn from the Mitochondrial MOA Seed Group Through the Structural Similarity Approach

Six drug compounds (i.e., nimodipine, nicardipine, nitrendipine, benidipine, methazolamide, and diethylcarbamazine) were also proved to wield the cell-protective ability against MPP⁺. Interestingly, the six compounds were able to protect cells from mitochondrial dysfunction as low as the range of 10 μM to 100 nM.

As shown in FIGS. 4(A) and 4(B), metabolic viability and cellular ATP were enhanced considerably by all six drug compounds with statistical significance.

As shown in FIG. 4(C), all of the drug compounds showed the effective range of dosages in which mitochondrial membrane potential increased more than the vehicle control, “MPP⁺” and the reference drug, RTA408.

As shown in FIGS. 4(D) and 4(E), nimodipine and nicardipine reduced ROS production significantly.

### SUMMARY

The inventors validated the efficacy of the fourteen (14) drug compounds that were selected through Standigm's AI technology on recovering mitochondrial function from damages and treating mitochondrial diseases employing an MPP⁺-lesioned human cell model. In the nanomolar range of treatment, all the drug compounds showed the increased survival ability and the ameliorated mitochondrial activities such as enhanced cell metabolic viability, increased intracellular ATP content, increased mitochondrial membrane potential, and reduced mitochondrial or intracellular ROS. In vitro and dose-response study of the fourteen (14) repurposed compounds indicate that the fourteen (14) compounds are promising therapeutics for mitochondrial diseases. Additionally, nimodipine and nicardipine showed that the drug compounds hold the protective activity on mitochondria and cells even under sub-nanomolar doses. This result would be beneficial to patients with mitochondrial diseases, who are taking high dose vitamins and health supplements. These results indicate that the active compounds discussed herein are useful to treat patients with mitochondrial diseases and/or enhance mitochondrial function in cells of patients.

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same results as the corresponding embodiments described herein may be utilized according to the presently disclosed subject matter. Accordingly, the appended claims are intended to include within their scope such process, machines, manufacture, compositions of matter, means, methods, or steps.

Various patents, patent application, publications, product descriptions, protocols, and sequence accession numbers are cited throughout this application, the inventions of which are incorporated herein by reference in their entireties for all purposes.

1. A method for enhancing mitochondrial function in a subject in need thereof, comprising administering to the subject an effective amount of one or more compounds selected from the group consisting of:
preladenant, cinepazide, mitiglinide, deforolimus, mycophenolate-mofetil, megestrol, lercanidipine, fusidic acid, nimodipine, nicardipine, nitrendipine, benidipine, methazolamide, diethylcarbamazine, and pharmaceutically acceptable salts thereof.

2. The method according to claim 1, wherein said one or more compounds are selected from the group consisting of:
nimodipine, nicardipine, and pharmaceutically acceptable salts thereof.

3. The method according to claim 1, wherein said enhancing mitochondrial function is one or more events selected from the group consisting of:
(i) enhancing cell viability;
(ii) increasing intracellular ATP content;
(iii) increasing mitochondrial membrane potential;
(iv) reducing mitochondrial reactive oxygen species (ROS); and
(v) reducing intracellular ROS,
compared to prior to the administering of the one or more compounds.

4. A method for treating a mitochondrial disease by enhancing mitochondrial function in a subject in need thereof, comprising administering to such subject an effective amount of one or more compounds selected from the group consisting of:
preladenant, cinepazide, mitiglinide, deforolimus, mycophenolate-mofetil, megestrol, lercanidipine, fusidic acid, nimodipine, nicardipine, nitrendipine, benidipine, methazolamide, diethylcarbamazine, and pharmaceutically acceptable salts thereof.

5. The method according to claim 4, wherein said one or more compounds are selected from the group consisting of:
nimodipine, nicardipine, and pharmaceutically acceptable salts thereof.

6. The method according to claim 4, wherein the mitochondrial disease comprises one or more selected from the group consisting of:
leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palmitoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-Arginine:Glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

7. A composition for enhancing mitochondrial function in a subject in need thereof and/or for treating a mitochondrial disease by enhancing mitochondrial function in a subject in need thereof, said composition comprising, as an active ingredient, an effective amount of one or more compounds selected from the group consisting of:
preladenant, cinepazide, mitiglinide, deforolimus, mycophenolate-mofetil, megestrol, lercanidipine, fusidic acid, nimodipine, nicardipine, nitrendipine, benidipine, methazolamide, diethylcarbamazine, and pharmaceutically acceptable salts thereof.

8. The composition according to claim 7, wherein said one or more compounds are selected from the group consisting of:
nimodipine, nicardipine, and pharmaceutically acceptable salts thereof.

9. The composition according to claim 7, wherein said enhancing mitochondrial function is one or more events selected from the group consisting of:
(i) enhancing cell viability;
(ii) increasing intracellular ATP content;
(iii) increasing mitochondrial membrane potential;
(iv) reducing mitochondrial reactive oxygen species (ROS); and
(v) reducing intracellular ROS,
compared to prior to the administering of the one or more compounds.

10. The composition according to claim 7, wherein the mitochondrial disease comprises one or more selected from the group consisting of:
leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palmitoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-Arginine:Glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.
